(19) United States
(12) Patent Application Publication
(10) Pub. No.: US 2026/0041860 A1
(43) Pub. Date: Feb. 12, 2026

(54) Breath-Enhanced Jet Nebulizer for HFNC Therapy

(71) Applicant: THE RESEARCH FOUNDATION FOR THE STATE UNIVERSITY OF NEW YORK, Albany, NY (US)

(72) Inventor: Gerald SMALDONE, Stony Brook, NJ (US)

(21) Appl. No.: 19/099,118

(22) PCT Filed: Jul. 28, 2023
(86) PCT No.: PCT/US2023/028975

§ 371 Date: Jan. 28, 2025

Related U.S. Application Data
(60) Provisional application No. 63/392,960, filed on Jul. 28, 2022.

(51) Int. Cl. A61M 11/06; A61M 16/00; A61M 16/06; A61M 16/10

(52) U.S. Cl. CPC A61M 11/06; A61M 16/0003; A61M 16/0666; A61M 16/109; A61M 2202/0007; A61M 2202/0208; A61M 2202/04; A61M 2205/3334; A61M 2205/3344; A61M 2210/0618

(57) ABSTRACT

A system and method for enhanced aerosol drug delivery during high flow nasal cannula (HFNC) therapy. The system comprises a breath enhanced jet nebulizer (BEJN) connected to a high flow medical breathing gas supply supplying breathing gas to the BEJN at flow rates of 5 L/min to 60 L/min, a nebulization gas supply, a drug infusion device that supplies a drug solution to the nebulizer for the delivery of nebulized drug to a patient. All the breathing gas and nebulized drug solution delivered to the patient passes through the BEJN. The rate of drug delivery increases with increases in the flow rate of the medical breathing gas supply and increases in infusion pump flow rates. The drug solution infusion rate is between 5 mL/hour and 50 mL/hour. This system and method enable efficient aerosol drug delivery to patients during HFNC therapy for prolonged periods, improving treatment outcomes.

### FIELD OF THE INVENTION

The present disclosure relates to a system and method for enhanced aerosol drug delivery during high flow nasal cannula therapy via a breath-enhanced jet nebulizer (BEJN).

### BACKGROUND OF THE INVENTION

In 2008, the potential for delivering aerosols of short acting drugs through infant, pediatric, and adult nasal cannulas was proposed utilizing a Solo™ vibrating mesh nebulizer (VMN) and bolus aerosol transport at 3 L/min through humidified nasal cannula systems. Over time, flows used in the clinical setting have increased to 60 L/min, defining the terminology high-flow nasal cannula (HFNC) therapy. Combining HFNC oxygen delivery with off-label delivery of aerosols via continuous infusion of a drug was then made practical by introduction of an infusion set administered via a drop-by-drop method.

A series of bench studies complemented by human deposition studies tested factors thought to be important in the delivery of nebulized aerosols via HFNC. Those studies indicated potential limitations in drug delivery particularly at gas flows commonly used in acutely ill adult patients ranging from 30-60 L/min. Bolus data (e.g., filling the nebulizer with a fixed volume) demonstrated significant aerosol losses in the circuit and, for continuous nebulization, limitations in nebulizer output.

Today, HFNC oxygen therapy is routinely used to treat patients with ARDS and other forms of respiratory failure. Typical gas flow through the cannula ranges from 30 to 60 L/min. Anecdotally, particularly during the COVID pandemic, clinicians have delivered pulmonary vasodilator aerosols to the lung in attempting to improve ventilation/perfusion mismatch by supplying oxygen and the vasodilating drug simultaneously. The potential benefits of this therapy include improved oxygenation, reduced dead space, improved ventilation/perfusion, and avoidance of intubation.

However, there may be limitations of certain nebulizing technologies to deliver particles through heated and humidified circuits, HFNC patient interface devices, and the nasal airways at the higher oxygen flows required for patients who are the sickest. For example, transnasal aerosol delivery is reduced at higher cannula gas flow. In addition, there is uncertainty in control of dosing with changing high-flow conditions. For clinical trials to answer questions with regard to treatment efficacy, the dose of the drug should be predictable and controllable.

For delivery of aerosols during HFNC, there are several studies that reported drug delivery for different nebulizer technologies. The results indicate that the higher the gas flow in the cannula, the lower the drug delivery. Losses in the tubing and nasal interface increase with increasing flow, and, for clinically relevant oxygen flows, conventional technology may not supply the patient with sufficient drug. In addition, the successful use of rapidly acting drugs often requires the precise adjustment of dose while clinical effects are monitored. Attempts to reduce tubing losses are inherently limited, and turbulence in the nasal turbinate cannot be easily modified. Aerosols leaking around the catheter-nasal interface are unpredictable. Once tubing limits are known, drug delivery can be increased simply by increasing the rate of drug infusion; however, conventional nebulizers have limited volumetric output rates.

One form of jet nebulizer, the breath enhanced jet nebulizer (BEJN), theoretically lends itself to high-flow therapy. Different from conventional nebulizers with fixed outputs, in the BEJN, the flow to the patient passes through the chimney of the nebulizer to enhance output. The magnitude of this effect is unknown during HFNC therapy. Breath enhanced jet nebulizers that may be useful in this invention are disclosed in PCT patent publication WO 2019/236896 A1.

A BEJN has a drug reservoir, a nebulization gas supply that drives a Venturi that draws a drug solution from the drug reservoir into a Venturi section that nebulizes the drug solution into atomized or nebulized droplets that efficiently deposit into small airways in the lungs when inhaled. A breathing gas supply enters the nebulizer through a chimney section. The breathing gas supply is the bulk of the breathing gases delivered to the patient. A distinguishing feature of a BEJN, as compared to a conventional jet nebulizer, is that a secondary constriction in the nebulizer enhances the Venturi effect and the rate of nebulization of the drug solution. In a particular type of BEJN, the reservoir is charged with a supply of a drug solution and the nebulizer is connected to a patient and the drug is nebulized until the reservoir is empty. In an alternative embodiment, for example used in this invention, a drug solution is added continuously to the reservoir during an extended course of treatment. A nebulizer that may be useful for continuous infusion of drug into the reservoir is disclosed in PCT patent publication WO 2022/140349 A1.

Patients with hypoxic respiratory failure are often treated with oxygen using HFNC. Delivery of aerosols by this route is uncertain and current practice confines treatment to intubated patients using continuous nebulization. The presently disclosed systems and methods provide a BEJN-HFNC route to treat critically ill patients before intubation thereby potentially avoiding any need for intubation at all. See M. McPeck, et al., In Vitro Model for Analysis of High-Flow Aerosol Delivery During Continuous Nebulization. Respiratory Care May 2023, respcare.10643, DOI: https://doi.org/10.4187/respcare.10643; and J. Moon, M. McPeck, J. Jayakumaran, G. Smaldone. Enhanced Aerosol Delivery during High Flow Nasal Cannula Therapy. Respiratory Care May 2023, respcare.10644, https://doi.org/10.4187/respcare.10644.

### SUMMARY OF THE INVENTION

Conventional transnasal aerosol delivery has limits on the amount of drug that can be nebulized and delivered to a patient at higher cannula gas flows that may be required to maintain sufficient oxygen for patients in severe respiratory distress. A new application of BEJN technology in HFNC can substantially increase the amount of drug that can be delivered transnasally. To test the use of a BEJN, the present disclosure measured aerosol outputs during continuous infusion by applying a humidified gas flow of up to 60 L/min directly to a prototype BEJN. Results were compared to conventional VMN (VMN) technology.

In another aspect, for HFNC continuous infusion aerosol therapy, infusion rates for vasodilators were calculated via a body weight-based protocol. Clinical response can be limited by aerosol losses and limitations of nebulizer output. Conventional nebulizers generate aerosol separate from high gas flow. BEJN technology combines the energies of nebulizer jet flow plus cannula high flow to generate aerosol and overcome these limitations. BEJN can therefore increase drug delivery beyond typical levels at the highest flows to a nasal cannula. The disclosure used real-time measurement of radiolabeled aerosol delivery to compare BEJN to a conventional VMN system in HFNC.

In another aspect, in vitro and in vivo assessment of transnasal aerosol delivery via HFNC were conducted. For in vitro studies, an anatomical nasal model was 3-D printed and validated. We simulated continuous infusion nebulization of short-acting drugs (albuterol/epoprostenol) during HFNC therapy to test the real-time gamma ratemeter technique with trans-nasal aerosol delivery to detect and measure Inhaled Mass (IM) dynamically during experiments with the 3-D printed nasal model.

Increasing gas flow increased BEJN output, which demonstrated the effects of breath enhancement. At 60 L/min, breath enhanced jet nebulizer delivered up to 5 times more aerosol compared with conventional VMN technology. BEJN delivered a wide range of dose rates at all high flows. In patients who are critically ill, breath enhanced jet nebulizer technology may allow titration of bedside dosing based on clinical response by simple adjustment of the infusion rate.

Therefore, based on the foregoing and continuing description, the subject invention in its various embodiments may comprise one or more of the following features in any non-mutually-exclusive combination:
A system for enhanced aerosol drug delivery during high flow nasal cannula therapy comprising a breath enhanced jet nebulizer (BEJN);
A system for enhanced aerosol drug delivery during high flow nasal cannula therapy comprising a medical breathing gas supply for supplying medical gas to the BEJN at a rate of 5 L/min to 60 L/min;
A system for enhanced aerosol drug delivery during high flow nasal cannula therapy comprising a high-flow medical breathing gas supply for supplying high-flow gas flow to the BEJN;
A system for enhanced aerosol drug delivery during high flow nasal cannula therapy wherein all the high-flow gas delivered to the patient passes through the BEJN.
A system for enhanced aerosol drug delivery during high flow nasal cannula therapy comprising a humidifier;
A system for enhanced aerosol drug delivery during high flow nasal cannula therapy wherein the BEJN is attached to a wet side of the humidifier or to a dry side of the humidifier;
A system for enhanced aerosol drug delivery during high flow nasal cannula therapy comprising a nasal cannula, wherein the humidifier is in fluid communication with the cannula for supplying aerosolized drugs to a patient;
A system for enhanced aerosol drug delivery during high flow nasal cannula therapy wherein infusion pump flow is between 5-50 mL/h;
A system for enhanced aerosol drug delivery during high flow nasal cannula therapy wherein the rate of drug delivery increases with increases in infusion pump flows higher than 5 mL/h;
A system for enhanced aerosol drug delivery during high flow nasal cannula therapy wherein the rate of drug delivery increases with increases in infusion pump flows between 5-50 mL/h at high-flow gas flow of less than 60 L/min at 50 psi;
A system for enhanced aerosol drug delivery during high flow nasal cannula therapy wherein all the high-flow gas flow first passes through the BEJN;
A system for enhanced aerosol drug delivery during high flow nasal cannula therapy wherein the flow of a drug to the BEJN to a high flow nasal cannula is between 2 and 50 mL/h;
A system for enhanced aerosol drug delivery during high flow nasal cannula therapy wherein the rate of drug delivery of the drug solution is proportional to the flow rate of the medical breathing gas supply;
A system for enhanced aerosol drug delivery during high flow nasal cannula therapy wherein the rate of drug delivery of the drug solution increases with increases in the medical gas breathing supply at a flow rate greater than 10 L/min;
A system for enhanced aerosol drug delivery during high flow nasal cannula therapy wherein the dose of drug is titrated over a period of hours by starting a low dose and increasing the dose of drug and flow rate of high flow breathing gas.
A system for enhanced aerosol drug delivery during high flow nasal cannula therapy wherein the patient is treated for a period of 30 minutes to 3 days or two hours to 24 hours; and
A method for enhanced aerosol drug delivery during high flow nasal cannula therapy comprising a breath enhanced jet nebulizer.

### BRIEF DESCRIPTION OF THE FIGURES

FIG. 1. Schematic diagram of system for enhanced aerosol drug delivery during high flow nasal cannula therapy.

FIG. 2. The rate of drug delivery (mg NaCl/min) for a vibrating mesh nebulizer (VMN) (black) and BEJN (white) at a high-flow gas rate of 60 L/min and various infusion pump flows (mL/h). Denotes an infusion pump flow in which the nebulizer filled with prolonged infusion (e.g., 1 h).

FIG. 3. The rate of test drug delivery (mg NaCl/min) for VMN (A) at infusion pump flows 5 mL/h to 20 mL/h and BEJN (B) at infusion pump flows 5 mL/h to 60 mL/h for the in vitro experiment, and high flow breathing gas supply at 10, 20, 30, 40 50, and 60 L/min. T Denotes the nebulizer filling during prolonged continuous nebulization. Brackets denote high-flow nasal cannula (HFNC) gas flow in L/min. Asterisks denote conditions in which infusion rate exceeded maximum for VMN and it was not tested

FIG. 4. Log aerodynamic diameter (mm) plotted against probability for aerosols from VMN (blue) and BEJN (black) at gas flows of 60 L/min. The infusion rate was 12 mL/h for the VMN and 20 mL/h for the BEJN. Mass median aerodynamic diameter at 50% probability±1 SD (shaded area).

FIG. 5. Ratemeter counts/min versus time (min) for a gas flow of 60 L/min at different infusion rates ranging from 10-40 mL/h. Aerosol output closely approximates a straight line for each condition, with the slope indicating the rate of aerosol delivery to the inhaled mass filter. For the BEJN, increases in infusion result in increases in slope at all infusion rates. For the VMN, the rate of delivery is essentially maximal at 12 mL/h with the nebulizer starting to fill at 20 mL/h, indicating that the rate of nebulization could not keep up with the flow of drug solution into the nebulizer. Aerosol output from mesh nebulizer increased with an infusion rate of 40 mL/h, but the nebulizer was full and leaking.

FIG. 6. Log of Deposition efficiency (%) as a scatter plot at the three designated infusion rates, 12 mL/hr, 20 mL/hr, 50 mL/hr.

FIG. 7. Deposition Rate (pgm NaCl) plotted against breathing frequency at each infusion rate with line of best fit. 12 mL/hr [closed circle], 20 mL/hr [closed triangle], 50 mL/hr [closed square]

### DETAILED DESCRIPTION OF THE INVENTION

To address the inherent limitations of delivery of aerosols during HFNC, the present disclosure relates to a system and method using a BEJN to increase aerosol output during HFNC. Specifically, the systems and methods according to the present disclosure are designed such that high-flow gas flow passes through the BEJN thereby synergistically increasing the output of the BEJN. This is the first proposal of such a solution that combines BEJN with HFNC wherein all of the high-flow breathing gasses passes through the BEJN via the chimney thereby increasing the output of the BEJN before being delivered to a patient. In conventional transnasal systems, the breathing gas supply to the patient bypasses the nebulizer. Moreover, as discussed below, during high flow therapy, the nebulizing capacity of prior art jet nebulizers and vibrating mesh nebulizers (VMN's) is substantially less than the BEJN used in this invention.

A further important feature of this invention is the capability for prolonged continuous nebulization while the dose to the patient is varied by changes in the rate of infusion into the nebulizer. The inventive system is capable of delivering variable dose rates of aerosolized drugs over hours to days.

A test protocol was designed to (1) define the maximum output of a BEJN (i-AIRE™ produced by InspiRx, Inc., Durham NC) receiving gas flows over the clinically relevant spectrum seen in the hospital (e.g., 5-60 L/min), and (2) measure aerosol delivery in vitro to a human model by using a newly developed 3-dimensional printed replica of an intact human nasal airway system. The BEJN system was compared with a conventional vibrating mesh system, which operated under the same conditions.

According to the present disclosure, increasing the rate of high-flow breathing gases to the BEJN directly increased BEJN aerosol output. At a drug infusion rate of 60 L/min, the presently disclosed system and method using a BEJN delivered up to 5 times more aerosol compared with conventional VMN technology. The BEJN delivered a wide range of dose rates at all high flows. In patients who are critically ill, the presently disclosed BEJN technology can allow titration of bedside dosing based on clinical response by simple adjustment of the infusion rate. The present invention provides as much as two orders of magnitude of variation in drug delivery rates, a substantial increase over conventional drug delivery technology.

The present invention can provide sufficient oxygen and drugs to patients in respiratory distress to avert the need for more invasive measures such as intubation on a mechanical ventilator. Conventional HFNC often fails to provide enough oxygen supply and high enough doses of drugs such as vasodilators and bronchodilators to avoid the need to intubate patients.

Breath enhancement increases drug output severalfold over conventional technology. BEJN can deliver drug at all relevant HFNC gas flows, facilitating a dose response assessment based on clinical needs at the highest HFNC gas flows by simply adjusting the infusion rate during therapy.

The drug dose can be titrated over a period of time, such as several hours to about 24 hours (for example two to 24 hours), in order to achieve a desired clinical response, such as an increase in oxygen saturation. The clinician can titrate the dug infusion and rate of aerosol production to that needed to achieve the desired clinical result, opening new avenues of treatment not available with HFNC and conventional nebulizers.

Additionally, patients can be treated with HFNC for an extended period of time of hours to days with this invention. For example, a patient could be treated on HFNC for 30 minutes to three days (or possibly more), or for two hours to 24 hours. This kind of extended treatment may not be possible with HFNC therapy using conventional nebulizers to deliver aerosolized drugs.

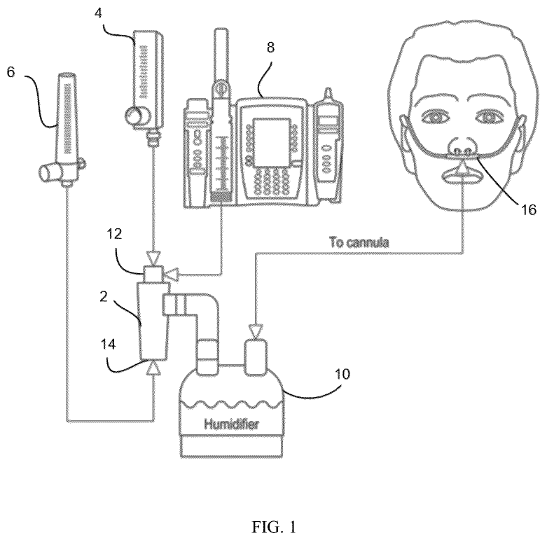

The inventive system and method employs a drug infusion device that adds a drug solution for nebulization to the nebulizer at a steady rate, for example 5 mL/hour to 50 mL/hour. The drug infusion device may be a pump (FIG. 1, reference no. 8). Alternative embodiments may be used, such as a syringe pump or an IV bag.

Throughout the disclosure the term breathing gas is not limited to any one type of gas and many types of gas are envisioned including air, oxygen (O₂), nitrogen (N₂), carbon dioxide (CO₂), hydrogen (H₂), and helium (He) and any various combinations of these and other gasses. The primary constituents of air are nitrogen (about 78%), oxygen (about 21%), and traces of other gases, including carbon dioxide, argon, and water vapor. In an aspect, a typical breathing gas is air, an oxygen enriched air, or pure medical oxygen at 50 psi.

The pressurized nebulizer gas supply may be the same blend of gas as the breathing gas or a different gas, such as air also at 50 psi. The nebulization gas supply, required to drive the Venturi that draws a drug solution into an orifice that atomizes (nebulizes) the drug solution, can operate at 2 L/min to 6 L/min. Typical flow rates may be, for example, 3 L/min or 5 L/min.

A humidifier is also required with a transnasal drug delivery system and method. In an embodiment, the nebulizer is on the dry side of the humidifier. The inventors found that losses in the humidifier are minimal with efficient nebulization. The nebulizer can also be on the wet side of the humidifier.

In an embodiment, a system is provided for enhanced aerosol drug delivery during high flow nasal cannula therapy with a breath-enhanced jet nebulizer (BEJN); a medical breathing gas supply for supplying breathing gas to a chimney of the BEJN at flow rates of between 5 L/min to 60 L/min at 50 psi; a nebulization gas supply of a medical breathing gas at between 2 L/min and 6 L/min at 50 psi; a drug infusion device that supplies drug solutions to the nebulizer at rates from 5 mL/hour to 50 mL/hour to supply a nebulized drug solution to the patient; a humidifier that humidifies the medical breathing gas supply; and a nasal cannula for delivering the medical breathing gas and nebulized drug solution to the nasal passages of a patient; wherein all the high-flow gas delivered to the patient passes through the BEJN. The BEJN may be attached to a dry side or wet side of the humidifier. The medical breathing gas supply may be air, an oxygen enriched air, or pure medical oxygen. The drug delivery device is a drug infusion pump or an intravenous (IV) bag. The rate of drug delivery of the drug solution is proportional to the flow rate of the medical breathing gas supply, and wherein the rate of drug delivery of the drug solution increases with increases in the medical gas breathing supply at a flow rate greater than 10 L/min. In an embodiment, the rate of the drug solution supplied to the nebulizer is increased with increased rate of the high flow breathing gas and maintained at a rate such that drug solution liquid does not accumulate in reservoir in the nebulizer.

In an embodiment, a method is provided for enhanced aerosol drug delivery during high flow nasal cannula therapy with a breath-enhanced jet nebulizer (BEJN); a medical breathing gas supply for supplying breathing gas to a chimney of the BEJN at flow rates of 5 L/min to 60 L/min, 50 psi; a nebulization gas supply of a medical breathing gas at between 2 L/min and 6 L/min at 50 psi; a drug infusion device that supplies drug solutions to the nebulizer at rates from 5 mL/hour to 50 mL/hour to supply a nebulized drug solution to the patient; a humidifier that humidifies the medical breathing gas supply; and a nasal cannula for delivering the medical breathing gas and nebulized drug solution to the nasal passages of a patient; wherein all the high-flow gas delivered to the patient passes through the BEJN.

The utility of the present invention is illustrated by the following studies demonstrating the utility of the invention.

Experiment 1. The Range of Nebulizer Outputs of the BEJN and VMN, at 60 L/Min, were Demonstrated.

We hypothesized that breath-enhanced nebulization would have a variable output affected by high flow. The limits of this function were unknown. A protocol was therefore designed to quantify the limits of output under changing conditions.

The principle of the drop-by-drop method of nebulization implies that, over time, no fluid should accumulate in the nebulizer. In this protocol, the maximum output of the breath-enhanced and mesh nebulizers was first defined by visual assessment. The protocol was designed to test the hypothesis that increasing high flow will increase nebulizer output. High-flow gas input was connected to the BEJN at the top of the nebulizer, so the interior of the nebulizer was affected by 2 flow sources, the high flow from the top at 50 psi gauge and the flow energizing the nebulizer from the bottom.

The nebulization gas supply can be in a range of 2 L/min to 6 L/min. In this experiment, the BEJN was operated at 5 L/min by using compressed air at 50 psi gauge; therefore, for a nominal gas flow of 60 L/min, the actual flow was 65 L/min. To equalize the high-flow gas rates, gas flow for the mesh nebulizer was adjusted by increasing the protocol flow by 5 L/min. The BEJN was interfaced to an infusion pump via a side infusion port. The VMN was connected to the high-flow circuit by using the T connector and to the infusion pump by the proprietary infusion port. The VMN was energized by the Pro-X controller.

At a given combination of gas flow and pump infusion rate, the nebulizers were observed for 20 min to define maximum infusion pump rates that generated a visual aerosol cloud without solution accumulating in the nebulizer. Based on these findings, the maximum infusion rates for each HFNC gas flow are outlined in Table 1.

TABLE 1
Maximum Infusion Flows for BEJN and VMN at Nominal
Gas Flows after 20 min of Observation

| | |
|---|---|
| BEJN | VMN |

| | Maximum | | Maximum |
|---|---|---|---|
| HFNC Flow, | Infusion Flow, | HFNC Flow, | Infusion Flow, |
| L/min | mL/h | L/mi | mL/h |
| 60 | 60 | 60 | 20 |
| 50 | 50 | 50 | 20 |
| 40 | 50 | 40 | 20 |
| 30 | 50 | 30 | 20 |
| 20 | 40 | 20 | 20 |
| 10 | 30 | 10 | 20 |

HFNC = high flow nasal cannula

With the data provided in Table 1, which outlines the limits of nebulizer function, the actual aerosol produced by the nebulizers was measured by using the setup described in FIG. 1. Components of FIG. 1 are as follows: BEJN 2, high-flow gas supply 4, gas supply device 6, drug delivery device 8, humidifier 10, chimney of the BEJN 12, bottom of the BEJN 14, cannula 16. This technique quantified output during high flow with only the humidifier as an added factor. The syringe pump used a 60-mL syringe filled with normal saline solution mixed with 3 mCi of ^{99m}Tc. The radioactivity of the prepared solutions was measured with a radioisotope calibrator, which defined the initial charge before starting each experiment.

Before the start of each experiment, the nebulizer was dry, empty, and free of radioactivity. The time at which this initial charge was measured served as the baseline time for decay correction of the subsequent measurements obtained throughout the experiment. The test nebulizer (BEJN or VMN) was attached to the dry side of a humidifier connected via a 6-inch hose to an output filter. The output filter consisted of 2 filters connected in series, a filter with removable media and a high-efficiency particulate air filter (bacterial/viral filter. This combination was attached to the outlet of the humidifier.

The output rate (counts/min) was measured for each infusion pump flow for 10-to-20-min intervals in real time by using a ratemeter. The ratemeter counts/min were converted to an output rate defined as mg NaCl/min. Based on the results listed in Table 1, at the clinical maximum gas flow of 60 L/min, saline solution mixed with ^{99m}Tc was infused at 5, 12, 20, 30, 40, 50, and 60 mL/h for the BEJN, and at 5, 12, 20, and 30 mL/h for the VMN. A single run was carried out at each infusion flow. For infusion pump flows of 5 mL/h, the pump was run for 20 min to allow the nebulizer to reach a steady state, otherwise all other infusion pump flows were run for 10 min. These data defined the maximum rates of aerosol generation in mg NaCl/min for each condition before being directed to a patient.

After demonstrating the range of nebulizer outputs for the BEJN and VMN, at 60 L/min, the nebulizers were attached to a ventilated model designed to assess aerosol delivery in an in vitro system that mimics HFNC therapy. This model is detailed in Experiment 2 whereas in vivo results are presented in Experiment 3, below. The setup is outlined in FIG. 1.

The HFNC humidifier circuit was connected to an HFNC interfaced to a 3-dimensional printed anatomically correct model of an adult head, which provided all ventilation through the nose. Collection filters were placed after the 3-dimensional printed head and connected to a piston pump for tidal ventilation. Activity on these filters was defined as inhaled mass (IM). The shielded ratemeter was positioned at the level of the IM filters for real-time measurement of radiolabeled aerosol accumulating on the filter. Aerosol reaching the filter complex represented particles that traversed all tubing and upper airways.

Tests were conducted using a single breathing pattern (tidal volume 750 mL, breathing frequency 30 breaths/min, and duty cycle 0.5), previously described as a distressed breathing Pattern. Two molded BEJN prototypes and 2 VMNs were used in rotation for all the experiments. The nebulizers were positioned in the circuit as described in FIG. 1. A saline solution that contained 4 to 6 mCi of ^{99m}Tc was drawn into a 60-mL syringe to achieve ^{99m}Tc concentrations of 67 to 100 mCi/mL.

Infusion rates over ranges, defined in Table 1, were tested for gas flows of 10, 20, 30, 40, 50, and 60 L/min. Testing each infusion/gas flow combination would take 2-3 h and provide several hundred data points. The complete range of experimental parameters were tested twice. The inhaled mass filter complex was changed every 80 to 100 min, and a new background count was obtained. After each experiment, the inhaled mass filter was measured by using the radioisotope calibrator to obtain the amount of radioactivity in mCi for a given count rate.

Filter data were used to calculate a conversion factor of ratemeter counts to mCi for each measurement. These data were converted to mg of salt (NaCl) based on the salt content of normal saline solution by using the formula:

$$μ g of {NaCl}_{time interval} = measured μ \text{} {Ci}_{time interval} / [ ( μ {Ci}_{syringe charge} ) / ( {mL}_{syringe charge} \times 9 , 000 μ g / mL ) ] .$$

which represents the amount of the drug being aerosolized and delivered during continuous nebulization. The mg of NaCl delivered to the output and inhaled mass filters was plotted as a function of time. The slope of each 10- or 20-min experimental condition represented the rate of drug delivery (mg NaCl/min). Examples for these tracings are included in Experiment 1, below.

The rate of drug delivery was analyzed by using multiple linear regression. Nebulizer technology, infusion pump flow, and HFNC flow were variables assessed. In a typical experiment, there were 5-10 data points for each condition used to define the slope that yields the output rate. Data were reported as mg NaCl/min. To allow statistical comparison, a multiple regression analysis was performed for experiments in which both nebulizers had a measurable output. The reported analysis included 48 data points, which represented 48 slopes. For some infusion rates, statistical comparisons were not possible, for example, for the experiments in which the VMN had no output. The magnitude of the regression coefficients define how much of the variability in the rate of drug delivery is ascribed to each independent parameter.

The mass median aerodynamic diameter of aerosol exiting the HFNC was determined by using a Marple-type 8-stage cascade impactor operated at 2 L/min. Normal saline solution mixed with ^{99m}Tc was infused at 12 mL/h for the VMN and 20 mL/h for the BEJN at gas flows of 60 L/min and sampled for 30 min. These infusion rates were chosen because rates of drug delivery at these infusion rates were similar. The ratemeter was used to measure the counts on the stages of the cascade impactor. Each experimental setting was run 3 times to ensure reproducibility. Activity on the cascade stages was plotted against probability to determine the mass median aerodynamic diameter.

### Results

Observational data for the first protocol are listed in Table 1. Analysis of these data illustrates effects of breath enhancement and defines the limits of nebulizer output. For each gas flow, the infusion pump flow was increased until fluid was seen filling the nebulizer, which indicated maximum output for that condition. For the BEJN, increasing the gas flow resulted in nebulizer outputs between 30 and 60 mL/h, with filling seen at 60 mL/h. For the VMN, changes in gas flow had no effect with the nebulizer beginning to fill at 20 mL/h for all gas flows.

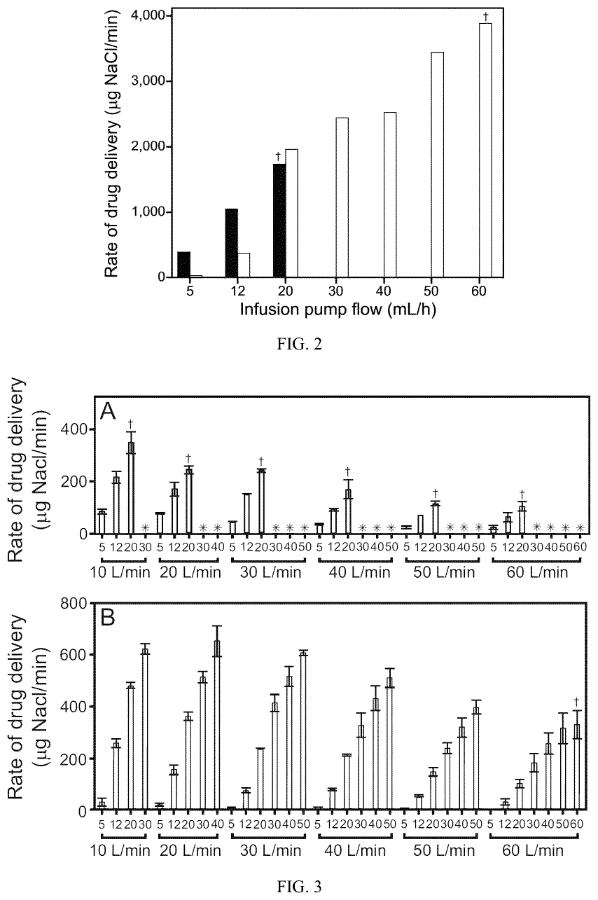

The rate of aerosol delivery to the output filter/min with increasing infusion flow is quantified in FIG. 2. All data were for a gas flow of 60 L/min. For the BEJN, aerosol output increased with each increment of infusion flow until, at 60 mL/h, the device started to fill. Therefore, its maximum output was an infusion rate of 50 mL/h. The measured output increased from 40.3 to 3,442 mg NaCl/min as infusion rates were increased. At the same gas flow, the VMN aerosol output ranged from 396.1 to 1,060 mg NaCl/min and reached a maximum at an infusion rate of 12 mL/h. The VMN began to fill at 20 mL/h. Infusion rates >20 mL/h resulted in rapid filling of the VMN, and aerosol outputs were not reported. Outputs marked with a cross in the figure denote infusion flows that resulted in filling. This protocol demonstrated the maximum potential for aerosol delivery at the highest practical gas flows before particles pass into the clinical delivery apparatus.

The results for the bench model described in FIG. 1 are shown in FIG. 3. These results measure the aerosol delivery rate to the trachea, the IM/min. Changes in the scale of the y axis (compared with FIG. 2) indicated that much of the aerosol measured in FIG. 3 had been lost, with the output values shown in FIG. 2, an order of magnitude greater than those in FIG. 3.

For each gas flow, IM/min increases with infusion flow, but the increase is limited for the VMN, which reaches maximum output at 12 mL/h. Beyond that rate of drug flow, the VMN was unable to nebulize at the same rate of drug infusion into the nebulizer, so the nebulizer began to fill with drug solution. BEJN output rates seemed similar to the VMN for infusion flows of 12 mL/h, but the BEJN was able to continue nebulizing efficiently at up to a drug infusion rate of 60 L/h. Regression analysis for these infusion flows were compared statistically. This analysis indicated similar function between the devices because nebulizer technology was not statistically important as a variable (Table 2).

TABLE 2
Multiple Linear Regression: Rate of Drug Delivery as a Function
of Nebulizer Type, Infusion Pump Flow and Gas Flow.

| Independent | | Standard | | | | |
|---|---|---|---|---|---|---|
| Variables | β | Error | 95% CI | P | R² | ΔR²* |
| Nebulizer | 26.2 | ±19.6 | −13.3 to 65.6 | 0.19 | 0.04 | NA |
| Type | | | | | | |
| Infusion | 12.5 | ±2.1 | 8.15 to 16.8 | <0.001 | 0.45 | 0.45 |
| Pump Flow | | | | | | |
| Gas Flow | −2.3 | ±0.27 | −2.86 to −1.75 | <0.001 | 0.79 | 0.34 |

*Represents the contribution to the total R value for each individual variable. NA = not applicable.

Multiple linear regression analysis of the rate of drug delivery indicated that 79% of the data was accounted for by this analysis (R2=0.79). Drug delivery was independent of nebulizer type (P=0.19) and dependent on infusion pump flow (P<0.001) and gas flow (P<0.001). For the rest of the conditions described in the figure, for example, infusion flows of 20-50 mL/h, only the BEJN produced aerosol with outputs that increased with increasing infusion flow up to 50 mL/h at all gas flows. In addition to the responsiveness of the nebulizers to infusion flow, the figure demonstrated that aerosol losses were greater at increasing gas flow. These losses are likely due to circuit losses and leaking at the nasal interface. At the higher gas flows often needed for those who are critically ill, the range of output for VMN was limited. For example, at 60 L/min, output rate ranged from 23.5 to 61.7 mg NaCl/min compared with the BEJN (3.16 to 316.8 mg NaCl/min).

FIG. 3A shows that for the VMN, the nebulization rate increased at drug infusion rates from 5 to 20 mL/h, but at the 20 mL/h level, the nebulizer began to fill with drug solution because the rate of nebulization in the VMN could not keep up with the drug infusion rate (denoted by T). The maximum drug infusion rate for the VMN for efficient nebulization was 12 mL/h.

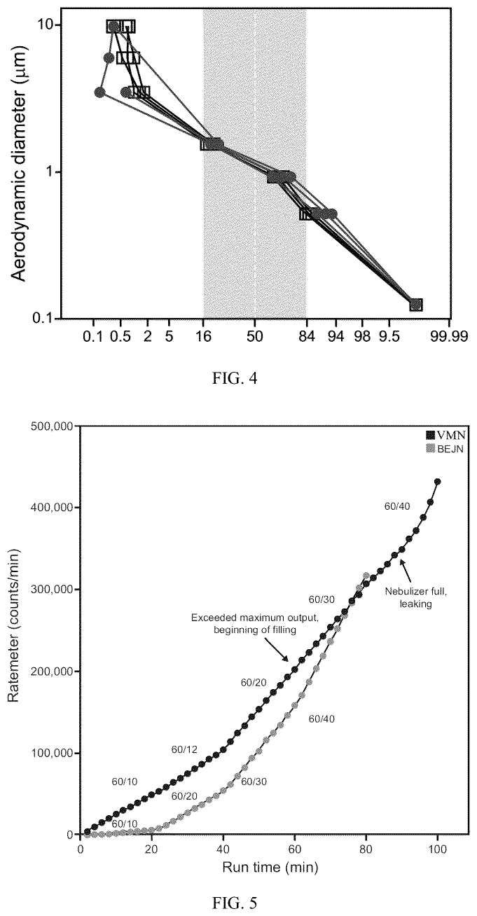

For all gas flows, BEJN was able to deliver more drug (FIG. 3B), and, despite circuit losses, analysis of the data in FIG. 3B shows that, at each gas flow, drug delivery could be adjusted over a wide range by changes in infusion rate. Particle size distributions are illustrated in FIG. 4. For the VMN, mass median aerodynamic diameter of 1.14±0.06 μm, with Geometric Standard Deviation (GSD)=1.37±0.07, and for the BEJN, mass median aerodynamic diameter of 1.10±0.03 μm, with GSD=1.41±0.04.

Moreover, FIG. 3B at the gas flow rate of 60 L/min demonstrates approximately two orders of magnitude range of drug delivery, i.e., from about 5 μg NaCl/min at an infusion rate of 5 mL/hour, to about 300 μg NaCl/min at an infusion rate of 60 mL/hour. This wide range of drug delivery, used at the highest breathing gas flow rate that would be used for the sickest patients, demonstrates significant advantages as a therapeutic modality.

### Discussion

This experiment demonstrates that during high-flow therapy, BEJN can increase aerosol output beyond that of conventional nebulizers. When interfaced with an HFNC circuit, some of the observed circuit losses can be balanced by increases in aerosol output facilitated by breath-enhanced nebulization. Comparing FIGS. 2 and 3 demonstrates that, in general, 90% of the aerosol generated is lost in the clinical circuit but as shown in FIG. 3, BEJN provides a greater range of aerosol delivery. The sensitivity of the BEJN to the infusion rate allows regulation of drug delivery over a wide range. Aerosol delivery can be adjusted over 2 orders of magnitude, flexibility that may allow titration of therapy based on clinical response. Both the VMN and BEJN allow some titration of therapy at the lower infusion rates and lower gas flows. The breath-enhanced device can function over a wider range, with increases in drug delivery at the bedside between 5 and 50 mL/h without having to increase drug concentration in the syringe or intravenous bag.

This study establishes a unique application of breath-enhanced technology. As shown in Table 1 and FIG. 2, the maximum output rates for the BEJN varied from 30 to 50 mL/h, depending on the high flow used. These values far exceed those reported in the general literature for typical nebulizers. According to the present disclosure, outputs usually approximate 10 mL/h and this value was measured for the BEJN during mechanical ventilation, which exposes the nebulizer to lower mean gas flows. The same device exposed to constant high flow generates 5 times as much aerosol.

Most bench studies, including this study, test devices over relatively short periods of time, especially when compared with a clinical situation that can require hours of continuous nebulization. To be clinically useful, a device should not fill. For some conditions, a 20-min observation period will not detect slow device filling. Prolonged testing of VMN and BEJN beyond 1h indicated that both devices tend to fill at the limits indicated in FIG. 2, which suggests that the practical infusion limit for VMN is 12 mL/h. Therefore, for the VMN to increase drug delivery, it would be necessary to increase the drug concentration.

Certain embodied studies according to the present disclosure mimicked a weight-based dosing regimen in which inhaled epoprostenol was delivered at different concentrations (7.5, 15 and 30 mg/mL) to a bench model designed to deliver 30 and 50 ng/kg/min for predicted body weights of 50,70, and 90 kg. The model used invasive ventilation with continuous nebulization of the VMN, which delivered epoprostenol to an IM filter over 20-min treatment periods. This weight-based dosing required higher infusion pump flows (12.0, 16.8, 21.6 mL/h).

It was observed that, for continuous nebulization, the maximum output of the VMN is closer to that reported in the company's service instructions of 12 mL/h. Clinicians should be aware that higher infusion rates may not yield proportionate increases in drug delivery. To ensure that a device can provide reliable continuous nebulization, it should be tested over periods that mimic actual clinical use, which could be hours. VMN infusion rates reported >12 mL/h have only been tested for 20 min. The regression analysis indicates that most of the variation in the data are due to infusion pump flow and gas flow (e.g., R²=0.79).

Nebulizer technology is not important for the conditions in which the devices will run continuously without filling, as shown in FIG. 3. These observations are predicted by the drop-by-drop method in which, in a steady state, all liquid infused into the nebulizer is nebulized. In addition to turbulent deposition in the delivery system, it is obvious from direct observation that large numbers of particles leak out around the nose as well as particles that are exhaled, even with nasal breathing.

Although the statistical analysis did not reveal significant differences between the nebulizers, inspection of FIG. 2 indicates that at the lowest infusion rates, the VMN output is greater than that of the BEJN.

These differences are reduced in FIG. 3. A likely explanation is that circuit losses are greater for VMN technology. When considering the limitations described above, at flows often used in those who are critically ill (30-60 L/min), the BEJN delivered 5 times more aerosol to the IM filter.

Reported clinical treatment plans for the off-label use of inhaled vasodilators have adapted a weight-based algorithm for deciding on dosing. Clinical response to continuous nebulization reveals that drugs fail to improve oxygenation 50% of the time. This suggests that weight-based dosing may be a limitation to adequate aerosol delivery to ensure patient improvement.

In such cases, it is considered that perhaps dosing should be increased until a desired physiologic outcome is attained or adverse effects are detected. To reach this point, the clinician should know that the device can deliver the drug.

BEJN offers an aerosol delivery device that can deliver the drug over a wide range at all clinically relevant oxygen high flows from very low doses to significant maxima. Only at the maximum point would the therapist have to change the solution to a higher concentration (e.g., an infusion rate of 50 mL/h, for a gas flow of 60 L/min). This would allow careful control and titration of drug delivery for infusion flows of 5-50 mL/h.

Experiment 2. The Nebulizers were Attached to a Ventilated Model Designed to Assess Aerosol Delivery in a System that Mimics HFNC Therapy.

This experimental method had the following specific objectives: (1) develop an adult human nasal airway model that is a realistic replica of human airways amenable to radionuclide assessment of deposition, (2) apply the real-time method of assessing delivery of radiolabeled aerosols to this model, and (3) validate the experimental setup by comparing deposition in the circuit and head model via vibrating mesh and BEJN (BEJN) technologies during continuous infusion aerosol delivery.

The experimental setup was incorporated into a fume hood to capture fugitive aerosols (FIG. 1). The 3D-printed head model, with intact, anatomically correct sinonasal air passages from the nares to the hypopharynx, was created from a computer file of a cast of an intact nasal airway system that was rendered from a CT scan of the head of an anonymous subject. An aerosol collection filter was designated as the inhaled mass filter to capture radio aerosol that transited the nasal airway of the head model.

The inhaled mass filter consisted of 2 filters connected in series, a PARI™ filter and a high-efficiency particulate air filter (bacterial/viral filter). The latter was used to capture any aerosol that passed through the PARI filter (5-10% leakage during high flow). The PARI filter is used because the filter media is removable and can be placed in a well counter to calibrate the ratemeter. The combined filters were connected via 18-inch×22-mm corrugated tubing to a port at the level of the hypopharynx of the head model. The other side of the inhaled mass filter was attached via 22-mm corrugated tubing to a piston ventilator used as a breathing simulator.

The gamma detector of a portable ratemeter (Model 2200 Scaler-Ratemeter), surrounded by a lead shield and mounted on an articulating arm, was extended into the hood from the outside and aligned with the inhaled mass filter as shown. Lead bricks were positioned around the inhaled mass filter and the gamma detector's shield to provide additional shielding to minimize artifactual background radiations from other high-activity sources (e.g., infusion syringe or intravenous [IV] bag) during experiments. Other equipment (gas sources, heated humidifier, infusion pump, and nebulizers) was positioned outside the fume hood.

The HFNC system consisted of an MR-850 heated humidifier chamber and controller plus an adult medium HFNC Optiflow™ (outer diameter 6.1 mm, inner diameter [ID] 5.1 mm) and heated-wire tubing set. Nebulizers were connected to the inlet side of the heated humidifier chamber. Four Solo nebulizers operated by the Aerogen™ Pro-X electronic controller in its continuous mode were used for vibrating mesh experiments. The VMN was compared with 2 molded prototypes of a novel BEJN (i-AIRE) that is being prepared for submission to the United States FDA for 510 (k) clearance.

Incorporating the chimney of the nebulizer directly in the high-flow circuit resulted in enhanced aerosol output during HFNC therapy. The BEJN was operated at 5 L/min from a 300-cu-ft cylinder of medical-grade air and a standard 0-15 L/min back pressure-compensated flow meter (Precision Medical, Northampton, Pennsylvania). A large, oil-free, portable air compressor with output pressure regulated to 50 psig was connected through a desiccant chamber and tubing to a high-flow, 0-75 L/min, back pressure-compensated flow meter to provide high air flow (up to 60 L/min) for experiments involving both nebulizer types.

Normal saline solution mixed with technetium sodium pertechnetate (^{99m}Tc-NS) was used as a surrogate for an actual drug such as albuterol or epoprostenol. The radiolabeled saline aerosol allows a real-time dynamic measurement of the inhaled mass as the radioactive aerosol accumulates over time on the inhaled mass filter during an experiment. The investigator triggers a 1-min reading of gamma counts accumulating on the inhaled mass filter at relevant intervals, for example, every 2 min for the first few minutes of an experiment, until a steady state is reached and thereafter at 5-min intervals. Simulated respiration was provided by a piston pump animal ventilator. A distressed breathing pattern (breathing frequency 30 breaths/min, tidal volume [VT] 750 mL, duty cycle [% inspiratory time] 0.50) was set on the respiration simulator based upon previously published studies.

To reduce background activity, the filter housing and shielded ratemeter were placed outside the lead brick shielding. The proximal side of the inhaled mass filter was attached to the connector on the hypopharynx of the head model by an 18-inch length of tubing, the length of which was necessary to connect the filter through the lead shielding. Aerosol presented via the HFNC was drawn through the head model, from nares to hypopharynx during the inspiratory stroke of the piston, which also drew aerosol into the inhaled mass filter where it deposited.

A programmable infusion pump system was used to control the infusion of ^{99m}Tc-NS into the nebulizers. For the BEJN, a BD 60-mL syringe with Luer-Lok tip and a proprietary tubing set connected to a 60-mL syringe pump or peristaltic IV pump with a 500-mL IV bag was used to infuse the nebulizer. For the VMN, the 60-mL infusion pump syringe and tubing set were used with the Alaris™ syringe pump due to the proprietary connector on the Solo nebulizer.

Several protocols were conducted to determine the suitability of this model for comprehensive bench testing. The proximity of the prepared infusion system served as a source of unacceptably high background radioactivity in the vicinity of the inhaled mass filter. To reduce this background radioactivity, the inhaled mass filter and the ratemeter's gamma detector were isolated and shielded by lead bricks between the inhaled mass filter and the head model.

During a prolonged experiment (e.g., >4 h), background radioactivity would increase. The goal was to keep this increase to <a few percent of total activity measured by the ratemeter. The shielding efficiency was tested by using a range of activity in the infusion setup (4-15 mCi), running experiments for several hours, and measuring residual radioactivity using the ratemeter in the vicinity of the inhaled mass filter.

The extended length of corrugated tubing required to reach through the lead shielding to connect the inhaled mass filter to the hypopharynx of the head model might affect aerosol delivery to the filter. To assess this possibility, an experiment was conducted to compare the use of no tubing (e.g., direct connection of the filter to the hypopharynx) or 18-inch and 36-inch sections of corrugated tubing between the head model and the filter. First, a clean inhaled mass filter was attached to the 22-mm ID port at the hypopharynx. A BEJN was filled with 6 mL normal saline radio-labeled with 5 mCi. Serial runs of 3 min of nebulized aerosol through the HFNC system were initiated first with the inhaled mass filter attached to the hypopharynx then 18 inches from the hypopharynx and 36 inches from the hypopharynx.

A solution containing 4-6 mCi of 99m Tc was drawn into a 60-mL syringe to achieve a ⁹⁹mTc concentration of 67-100 mCi/mL. The radioactivity of the prepared solution was measured with a radioisotope calibrator defining the initial syringe charge before starting the experiment. Saline mixed with ^{99m}Tc was infused continuously by 60-mL syringe pump or 500-mL IV bag pump into the BEJN or VMN at a rate of 10-20 mL/h. Gas flow to the HFNC was 60 L/min. For the BEJN, the gas entered through the top of the nebulizer chimney, for the VMN through the standard nebulizer T connector. Total gas flow for the BEJN was adjusted for the 5 L/min supplied to the bottom of the nebulizer. After nebulization was complete, all components including the head model were imaged by gamma scintigraphy to measure deposition of radioactive aerosol throughout the system to determine the mass balance.

TABLE 3
Mass Balance (% of Infused Technetium-99m NS) for i-AIRE
and Solo Nebulizers in High-Flow Nasal Cannula Circuit.

| | P | i-AIRE | Solo |
|---|---|---|---|
| Recovery | >0.99 | 75.91 ± 4.27 | 75.50 ± 3.01 |
| Nebulizer residual | 0.006 | 32.17 ± 4.11 | 3.83 ± 1.08 |
| IM filter | 0.79 | 6.09 ± 1.15 | 5.78 ± 1.18 |
| T/elbow | 0.006 | 0.83 ± 0.15 | 3.60 ± 0.19 |
| Humidifier | 0.01 | 4.66 ± 1.75 | 17.43 ± 2.87 |
| Circuit | 0.006 | 6.34 ± 0.96 | 13.69 ± 1.41 |
| HFNC | 0.53 | 14.61 ± 1.25 | 17.64 ± 3.21 |
| 3D-printed head | 0.53 | 11.13 ± 2.13 | 12.88 ± 1.75 |

Data are expressed as mean ± standard error. P values compare devices. IM = inhaled mass. HFNC = high-flow nasal cannula. 3D = 3 dimensional.

For the mass balance data, results from 4 VMN and 7 BEJN infusion experiments were reported. Slopes of ratemeter data points for a given condition were defined by linear regression and expressed as CPM/min. Mass balance data for the nebulizers were compared by the Mann-Whitney non-parametric test.

### Results

Serial aerosol delivery, as a percentage of the nebulizer charge, to inhaled mass filters located at the exit of the head model 18 inches from the model and 36 inches from the model was 3.0, 3.0, and 1.9%, respectfully, for 3-min runs of an i-AIRE nebulizer. These data indicated that moving the filter 18 inches from the head position to prevent background contamination had no effect on measured aerosol delivery to the inhaled mass filter.

In the absence of any shielding, the typical count rate for 15 mCi radioactivity in the infusion syringe resulted in a background count rate of >500,000 CPM after several hours of nebulization. With the shielding in place, the count rate was reduced to about 5,000 CPM. After 4 h of study, the ambient background count rate in the region of the filter averaged 1% of the total activity sampled on the inhaled mass filter.

Mass balance results are shown in Table 3. The major source of aerosol loss was at the nasal interface, with simi-lar losses to the environment for each device (25% lost after accounting for all other activity). Significant differences in deposition on circuit components were seen between nebulizers. The nebulizer residual was higher for the BEJN (P=0.006), whereas circuit losses, including humidifier, were higher for the VMN (P=0.006).

There were no differences in delivery to the filter and head model. Losses in the head model were confined largely to the nasal cavity (85% of head deposition). Once particles made it past the nose, there was little deposition in the rest of the airway. FIG. 5 illustrates the real-time capture of radioactivity for single examples of the nebulizers on the inhaled mass filter. Decay-corrected activity plotted as CPM versus time in minutes is shown for a single gas flow of 60 L/min. Infusion flow was increased in increments from 10-40 mL/h. For the fixed gas flow, as infusion flow was increased, the rate of aerosol delivery increased as evidenced by the slopes of the activity lines for each infusion rate. The slopes of the output lines are tabulated in Table 4. For the BEJN, aerosol delivery rates in CPM/min incrementally increased with obvious slope changes with each infusion change. Slopes ranged from 338-8,111, which is a 24-fold increase.

For the VMN, the initial rate of aerosol delivery was much higher at the lowest infusion rate (2,467). There was a small increase in slope at the infusion rate of 12 (the suggested maximum in the Aerogen manual). Further increases in infusion rate to mesh nebulizer resulted in complex changes in aerosol delivery, e.g., linear between rates of 12-20 mL/h, minimal increase between 20-30, and a curvilinear change from 30-40. During the infusion at 20 mL/h, the mesh nebulizer was observed to begin filling, indicating that it had exceeded maximal out-put, supported by the lack of effect on slope from 20-30. At the infusion rate of 40 mL/h, the nebulizer was observed to be full and leaking.

### Discussion

This experiment describes an anatomically correct nasal oropharyngeal airway installed in a circuit designed to test HFNC aerosol delivery in real time during continuous nebulization. Aerosols passed through the head model with minimal deposition except at the nasal orifice.

TABLE 4
Drug Delivery Rates for i-AIRE and Solo Nebulizers
at Different Continuous Rates of Infusion.

| Infusion Pump Flow mL/h | i-AIRE | Solo |
|---|---|---|
| 10 | 338 | 2,467 |
| 12 | NM | 2,828 |
| 20 | 2,575 | 4,918* |
| 30 | 5,311 | 5,106* |
| 40 | 8,111 | 6,187* |

I-AIRE and Solo are ratemeter counts CPM/min.
*indicates nebulizer filling.
NM = Not measured.

The mass balance data demonstrated deposition patterns in the circuit that revealed significant differences between devices but similar delivery to the inhaled mass filter. The VMN had minimal residual compared to the BEJN but more deposition in the circuit and humidifier. These losses balanced each other as there were no differences in aerosol deposition in the head model (12%) and inhaled mass filter (6%).

High-flow cannula and the nasal passages are often suspected as barriers to therapy with possible turbulent impaction losses. However, this data indicate that a limiting factor of a typical HFNC setup is the leaking of aerosol around the nares and exhaled particles during expiration (25%). These losses cannot readily be prevented by cannula design.

In the example shown in FIG. 5, the activity was reduced by 29% (or <1% of the total mass balance) after cleaning the face. Different breathing patterns will likely yield different quantitative measurements. In their study, the distressed pattern with the largest VT delivered the most drug.

Mass balance data provide insight as to the behavior of circuit components during aerosol delivery, but for critically ill patients on HFNC therapy, the rate of drug delivery is likely more important to clinical response than a single value of deposition on the inhaled mass filter, a possible advantage of the real-time analysis. The drop-by-drop method of delivering aerosols by continuous nebulization provides a steady rate of drug to the airway that can be titrated to a desired response. An understanding of how to best do this depends on knowledge of the responsiveness of the aerosol delivery system, e.g., how aerosol delivery changes when circuit parameters are varied.

The present disclosure demonstrates how the real-time ratemeter technique can be applied to HFNC aerosol delivery. Ratemeter data demonstrated that the experimental setup was sensitive to infusion flow and that the individual nebulizer technologies behaved differently. For example, in Table 4 and FIG. 5 at 60 L/min gas flow and infusion rate of 10 mL/h, the VMN delivered aerosol at a much higher rate than the BEJN. Those differences reversed as the infusion rate was increased. Inspection of FIG. 5 indicates the slope of the VMN was less responsive to increases in infusion rate, suggesting that it was at or near maximum output starting at 20 mL/h. However, even at this rate, the nebulizer began to fill with drug solution since the rate of nebulization could not keep up with rate of drug infusion. FIG. 5 provides further support that the maximal nebulization rate of the VMN was about 12 mL/h, because the slope of the VMN line did not change from 12 mL/h to 40 mL/h.

In concert with the slope changes, direct observation of the nebulizer indicated that it was filling. For the drop-by-drop method to function over time, the nebulizer should not fill. The rate of nebulization should be equal to the rate of infusion of drug solution into the nebulizer, so if the nebulizer reservoir starts to fill, the rate of nebulization is insufficient to keep up with the rate of addition of drug to the nebulizer. This is undesirable. The point in the graph where the nebulizer started to fill is an indicator of maximal output. At 40 mL/h, output from the VMN increased with an upward curve, which correlated with this device being full and leaking. A likely explanation for the curvilinear behavior is increased pressure from the infusion pump resulting in increasing output.

The mass balance data according to the present disclosure suggest that one approach to increasing aerosol delivery would be to balance the losses by increasing nebulizer output. It is believed that the BEJN, a device responsive to gas flow through it, increases output sufficiently to increase delivery to the lungs in spite of existing losses in the circuit. The present disclosure outlines basic parameters defining aerosol delivery over a wide range of conditions (e.g., multiple rates of gas flow and infusion rates) that may be relevant to the design of therapeutic protocols using HFNC therapy to deliver aerosols in clinically relevant situations.

### Experiment 3. In Vivo Experiment to Measure Regulated Aerosol Delivery to the Lungs of Normal Volunteers Using a Nebulizer Designed to Overcome the Limitations of HFNC Therapy.

This was a clinical trial with healthy volunteers designed to measure drug deposition in the lungs (using a test solution) using a BEJN and HFNC. The protocol measured serial deposition of radiolabeled test aerosols in the lungs of normal volunteers. The delivery of the aerosol was controlled by a syringe pump with the goal to deliver drug to the lung at a precisely controlled predictable rate.

Nine healthy adult volunteers were enrolled in the study and signed an IRB-approved consent form. After baseline spirometry, subjects were seated in front of a gamma camera and a fifteen min background image obtained with the camera set for 99mTechnetium (^{99m}Tc). Following background imaging, subjects held a double walled Lucite container with a circular space between the walls filled with approximately 5 mCi ^{99m}Tc diffused throughout and a two-minute transmission image was obtained. The transmission image was utilized to aid in optimal positioning of the subject and to estimate regional lung volume for use in regional lung deposition calculations.

Normal saline was used as a marker of a test drug. Two test solutions were prepared. Non-radioactive “cold” saline in a 1-liter IV bag was hung on one side of an Alaris infusion pump. On the other side, equipped with a syringe infusion pump, radio labeled normal saline was mixed with 99mTechnetium bound to Diethylenetriamine pentaacetate, (^{99m}Tc-DTPA) to create a radioactive solution with approximately 3.5 mCi/mL. High concentrations of ^{99m}Tc-DTPA were used to overcome anticipated losses in the HFNC circuit.

Aerosol inhalation was performed. Volunteers were outfitted with a high flow nasal cannula and exposed to a test airflow of 20 L/min that was gradually increased to 60 L/min over 5-10 min. In addition to the test air flow, a saline infusion using the IV bag introduced cold saline into the nebulizer (i-AIRE, with a nebulizer gas flow of 5 L/min air at 50 PSIG). The cold saline was infused at increasing rates from 20 to 50 mL/hr over 10 min. Volunteers were instructed to breathe normally via the nose. Once comfortable on a gas flow of 60 L/min and infusion rate of 50 mL/hr, the infusion was switched to the syringe side of the pump and a radio-labeled continuous infusion was initiated at 12 mL/hr, a rate that would provide a low rate of aerosol delivery to the airway.

The number of tidal breaths was counted over the duration radio labelled infusions (frequency of breathing (breaths/min). Subjects were monitored with the gamma camera, and once the count rate of the image was sufficient for accurate scanning, the infusion was stopped, and a five-minute static image taken. With image acquisition complete, the infusion protocol was repeated at 20 mL/hr and 50 mL/hr. Each run from start of infusion to completion of the static scan took approximately 10 min. After completion of the last lung image, a lateral scan of the head was performed to measure nasal deposition.

### Analysis

Deposited radioactivity was quantified by normalizing count rates for each image to one minute (CPM). Activity from both lungs was included in all calculations because most upper airway activity remained in the nose and was not swallowed. After correction for room background on the first deposition image, succeeding deposition images were corrected by subtraction of the activity from the preceding image and decay corrected. Deposition (CPM) was converted to μCi. Chest wall attenuation was estimated using equation (1),

$${AF}_{gm} = 0.0562 BMI + 0.907 , \qquad ( 1 )$$

where AF_{gm} represents attenuation factor for geometric mean, BMI for body mass index in kg/m².

Deposition rate expressed in μgm NaCl/min based on the salt content of normal saline (9000 μgm/L) for each infusion rate was calculated via equation (2),

$$DR = \frac{A * AF}{E * t} / \frac{SC}{TV * 9000} , \qquad ( 2 )$$

where DR is deposition rate in μgm NaCl/min, A is activity deposited (counts) normalized to one minute that has been background and decay corrected, E is camera efficiency (counts/μCi, measured with activity placed on camera face), t is total time of infusion (min), SC is syringe charge (μCi), and TV is total volume of solution (mL).

Camera efficiency was measured using a Pari filter infused with a known amount of ^{99m}Tc-DTPA each experimental day. The DR represents the lung dose of drug/min. With the lung dose calculated, the efficiency of drug delivery (DE) for the high flow nasal cannula system could be calculated by equation (3),

$$DE \% = [ DR ( μçi / min ) / mCi infused ] * 100. \qquad ( 3 )$$

The DE represents the percentage of drug, when compared to total infused, that is deposited in the lung. Nasogastric deposition efficiency, consisting of cumulative nasal and stomach deposition for the entire experiment, was estimated in a similar fashion. During lateral imaging of the head, the subject's nose was essentially on the camera face and deposited activity was measured as counts/E. For small amounts of stomach activity, radioactivity attenuation correction across individuals was estimated to be double that of the lung (2AF).

Regional deposition was quantified using central to peripheral ratios (C/P). Whole lung and central regions were hand drawn around the final deposition image and superimposed on the transmission image. The central region of interest encompassed approximately ⅓ of the lung containing the central airways. The ratio of counts in the central to peripheral region for the deposited aerosol particles (aC/P) was normalized by the transmission image ratio (tC/P) to correct for regional lung volume resulting in the ratio of deposited particles per unit of lung volume (sC/P). Using this technique, sC/P of 1.0 represents particles deposited in small airways and alveoli.

Statistical significance between groups was determined using the Wilcoxon test. Results are reported as a mean±standard deviation.

### Results

Baseline lung function and anthropomorphic details of the test subjects are listed in Table 5. An example of serial deposition images for a test subject at the three test infusion rates is shown in FIG. 6. Counts on the 5 min static images increase sequentially with 69.7 μCi, 142.6 μCi, and 348.9 μCi in the 12 ml/hr, 20 ml/hr, and 50 ml/hr images respectively.

TABLE 5
Baseline characteristics of subjects including sex, age, FEV1
and FVC reported as percent of predicted, and FEV1/FVC ratio

| Patient ID | Sex | Age | FEV₁ (%) | FVC (%) | FEV₁/FVC |
|---|---|---|---|---|---|
| 1 | M | 75 | 115% | 103% | 0.81 |
| 2 | F | 74 | 84% | 90% | 0.70 |
| 3 | F | 59 | 92% | 93% | 0.77 |
| 4 | F | 36 | 123% | 130% | 0.78 |
| 5 | M | 60 | 101% | 97% | 0.81 |
| 6 | F | 31 | 100% | 94% | 0.89 |
| 7 | M | 61 | 86% | 84% | 0.80 |
| 8 | M | 73 | 98% | 100% | 0.71 |
| 9 | F | 72 | 112% | 97% | 0.87 |

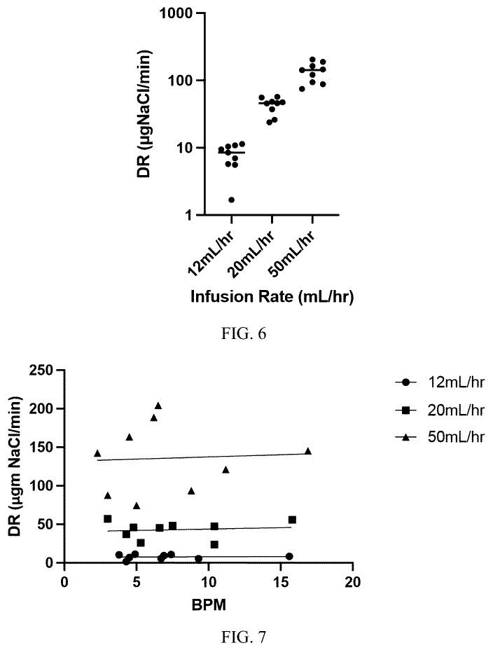

In general, the lungs were well defined with minimal stomach activity unless some nasal activity was swallowed towards the end of the test protocol. The images qualitatively suggest peripheral deposition with no activity seen in central airways. FIG. 7 describes the individual deposition rates, expressed as μgm NaCl/min at each infusion rate plotted on a log scale to bring out details. Mean deposition rates (7.84±3.15 μgm NaCl/min, 43.0±11.8 μg NaCl/min, 136±45.3 μgm NaCl/min) increased significantly with infusion rate e.g., 12 to 20 mL/hr (P=0.0039) and 20 to 50 mL/hr (P=0.0039).

Table 6 displays the deposition efficiency (%) of the lungs for each infusion rate and combined nasogastric efficiency for measured at the end of the experiment. Deposition efficiency significantly increased between 12 mL/hr compared to the higher infusion rates e.g., 0.44±0.18% at vs 1.45±0.40% at 20 mL/hr and 1.82±0.61% at 50 mL/hr (P=0.0039). Differences between deposition efficiency at 20 and 50 mL/hr were not significant (P.0742). On average, 6.23±1.9 of total infused radioactivity from the syringe pump deposited in the nasogastric region (5.95±1.57% nasal/0.28±0.51% stomach).

TABLE 6
Deposition efficiency (%) at each infusion rate and nasogastric
efficiency (%) measured at end of experiment for each
subject followed by mean ± standard deviation

| | 12 mL/hr Eff | 20 mL/hr Eff | 50 mL/hr Eff | Nasogastric Eff |
|---|---|---|---|---|
| Patient ID | (%) | (%) | (%) | (%) |
| 1 | 0.33 | 1.55 | 1.60 | 6.75 |
| 2 | 0.09 | 0.87 | 1.01 | 5.58 |
| 3 | 0.39 | 1.27 | 1.94 | 5.67 |
| 4 | 0.61 | 1.95 | 1.15 | 6.31 |
| 5 | 0.53 | 1.50 | 2.57 | 6.75 |
| 6 | 0.63 | 1.57 | 2.14 | 4.38 |
| 7 | 0.49 | 1.88 | 1.96 | 8.76 |
| 8 | 0.31 | 0.82 | 1.25 | 8.5 |
| 9 | 0.60 | 1.65 | 2.73 | 4.61 |
| Mean ± SD | 0.44 ± 0.18 | 1.45 ± 0.40 | 1.82 ± 0.61 | 6.37 ± 1.53 |

Regional lung deposition (Table 7), represented by C/P ratios normalized for volume (sC/P ratio) averaged 1.04±0.31. FIG. 7 illustrates the relation between deposition rate (in μgm NaCl/min) and respiratory frequency at the various infusion rates. While the effects of changes in infusion rate are apparent, there was no correlation between x rate of deposition and breathing frequency; P=0.885, R²=0.0032 at 12 mL/hr, P=0.753, R²=0.015 at 20 mL/hr, and P=0.884, R²=0.0033 at 50 mL/hr.

TABLE 7
Central to peripheral ratio reported as actual ratio,
transmission image ratio, and normalized ratio.

| Subject ID | aC/P | tC/P | sC/P |
|---|---|---|---|
| 1 | 0.290 | 0.313 | 1.041 |
| 2 | 0.487 | 0.560 | 0.870 |
| 3 | 0.520 | 0.444 | 1.161 |
| 4* | 0.489 | 0.661 | 0.740 |
| 5 | 0.451 | 0.415 | 1.081 |
| 6 | 0.371 | 0.301 | 1.259 |
| 7* | 0.446 | 0.559 | 0.798 |
| 8* | 0.420 | 0.757 | 0.555 |
| 9 | 0.614 | 0.446 | 1.375 |
| Mean ± SD | 0.439 ± 0.138 | 0.462 ± 0.146 | 1.04 ± 0.307 |

*one lung not included due to stomach or esophageal deposition

### Discussion

This study demonstrates that HFNC therapy can be used to target the lungs with aerosol. Delivery to the peripheral airways can be controlled over a wide range of deposition rates defined by the rate of infusion. As shown in FIG. 6, rates of drug delivery ranged over an order of magnitude. Deposition rates per se reflect the dose to the lungs per min. For fast acting drugs (e.g., epoprostenol) clinical responses could be assessed after reaching a steady state for each infusion rate and adjusted up or down as needed. For drugs with longer half-lives such as antibiotics, clinically relevant lung doses could be delivered using an appropriate mean delivery rate for a fixed number of minutes (e.g., dose rate (t)=total lung dose).

While there were some differences in deposition rate from patient to patient, the variability is much less than that reported for typical aerosol delivery of small particles for tidal breathing, where differences in breathing frequency can result in significant changes in deposition. (Bennett). During HFNC therapy, contrary to observations in spontaneously breathing normal subjects, deposition was not sensitive to breathing frequency.

Fine particles inhaled during tidal breathing deposit primarily by gravitation settling, governed by particle residence time in the airways which is a function of breathing frequency. Deposition during HFNC therapy may be governed by different mechanisms. With HFNC therapy, dead space is effectively reduced, and particles may be transported to the peripheral lung by convection created by the high flow gases which are full of aerosol particles. Deposition in the distal lung may be determined by an exchange between the aerosol front and alveolar gas, a process that may be less sensitive to tidal breathing.

Significantly, the normalized mean C/P ratio close to 1.0 (Table 7, bottom row, right) indicates that the test drug was largely deposited in the alveoli and small airways of the lungs, which is desirable.

Consistent with this hypothesis, the C/P ratios in these subjects were indicative of deposition in the smallest airways and alveoli. Further work is necessary to understand particle transport within the lungs during HFNC therapy to assess its potential for targeting small airways, particularly in disease.

While lung deposition was controlled, the rate of drug delivery to the lungs was much less than delivery to the nose. We did not record serial nasal delivery for each infusion rate, but for the total experiment the deposition efficiency of the nose was approximately 10 times higher. In future studies testing active drugs, systemic drug effects may be affected by this observation.

This protocol tested HFNC delivery simulating the most difficult clinical conditions for the critically ill patient, e.g., requiring 60 L/min of gas. However, these data apply to normal subjects and the behavior of aerosols in vivo remains to be demonstrated in patients with lung disease. HFNC aerosol delivery is inefficient, with significant losses across the circuit as well as at the nasal interface. Over 95% of the infused drug is lost, as illustrated by the deposition efficiencies reported in Table 6. Breath enhanced nebulization overcomes the inefficiencies of nasal delivery. Without increases in nebulizer output with increases in infusion rate, the losses in the nasal circuit, including nasal deposition, would be difficult to overcome.

### Overall Conclusions

The present disclosure shows that large amounts of drug can be delivered via continuous infusion nebulization at high flow rates used in HFNC therapy. The amount of drug being delivered can be controlled. The rate of drug delivery is dependent on nebulizer type, infusion pump flow, and breathing gas flow rate. The present system and method using a BEJN has superior results including higher rates of drug delivery at higher flow rates compared to conventional VMN technology.

BEJN produces increasing aerosol with increasing gas flow in a model of HFNC delivery. This study outlines conditions that may provide a therapeutic dose of vasodilators and other important drugs to the patient who requires high flows of oxygen. The present disclosure also shows that HFNC therapy can deliver controlled amounts of aerosol particles to the distal airways of human subjects.

According to certain non-limiting embodiments, the system and method according to the present disclosure is designed to work with any type of drug capable of being nebulized, especially those destined for the lungs and other breathing passages or those where it would be beneficial to deliver as such. Currently there are two short acting drug classes that are readily amenable to HFNC delivery, both able to be given continuously. These include albuterol and prostaglandins e.g., epoprostenol. Certain of these include but are not limited to: Veletri (epoprostenol), Flolan (epoprostenol), Remodulin (reprostinil), Tyvaso (reprostinil), and Ventavis (iloprost), common short-acting bronchodilators include such as salbutamol and levalbuterol, short-acting anticholinergics such as ipratropium bromide, and short-acting vasodilators such as nitroglycerin.

The present disclosure is also envisioned for application with long-acting drugs which can be given via HFNC over fixed periods of time such as, for example, antibiotics and steroids. Certain of these include but are not limited to: long-acting bronchodilators such as long-acting beta-agonists (LABAs) like salmeterol and formoterol, and long-acting anticholinergics like tiotropium and aclidinium; inhaled corticosteroids (ICS) such as fluticasone, budesonide, and mometasone; and long-acting vasodilators such as long-acting nitrates like isosorbide mononitrate.

According to certain non-limiting embodiments, the drug delivery device can be a drug infusion pump, an IV bag, or similar acceptable device for providing a drug to the BEJN.

According to certain embodiments, the BEJN is attached to the dry side of the humidifier; however, other configurations are also envisioned wherein the BEJN is attached to the wet side of the humidifier.

With dose being a function of the dose rate (μgm/min-regulated by the infusion pump) times the time (min) resulting in μgm deposited, as compared to conventional nebulizers, the ability to dose with the presently disclosed method and system is more controllable and better defined and, therefore, more amenable than conventional means of drug delivery during HFNC.

1. A system for enhanced aerosol drug delivery during high flow nasal cannula therapy comprising:
a breath-enhanced jet nebulizer (BEJN) that nebulizes a drug solution in a reservoir in the BEJN;
a medical breathing gas supply for supplying high flow breathing gas to a chimney of the BEJN at flow rates of between 5 L/min to 60 L/min at 50 psi;
a nebulization gas supply of a medical breathing gas at between 2 L/min and 6 L/min at 50 psi;
a drug infusion device that supplies drug solutions to the nebulizer at rates from 5 mL/hour to 50 mL/hour to supply a nebulized drug solution to a patient;
a humidifier that humidifies the medical breathing gas supply; and
a nasal cannula for delivering the medical breathing gas and nebulized drug solution to the nasal passages of a patient;
wherein all the high-flow breathing gas delivered to the patient passes through the BEJN; and
wherein the rate of nebulization increases with the rate of medical gas breathing supply at a flow rate greater than 10 L/min.

2. The system of claim 1, wherein the BEJN is attached to a dry side of the humidifier.

3. (canceled)

4. The system of claim 1, wherein the medical breathing gas supply is air, an oxygen enriched air, or pure medical oxygen.

5. The system of claim 1, wherein the drug delivery device is a drug infusion pump or an intravenous (IV) bag.

6. (canceled)

7. The system of claim 1, wherein the rate of the drug solution supplied to the BEJN is increased with increased rate of breathing gas and maintained at a rate such that drug solution liquid does not accumulate in the reservoir in the BEJN.

8. The system of claim 1, wherein the rate of infusion of drug solution into the BEJN is titrated over a period of hours between a dose of 5 mL/hr to 50 mL/hr by starting at a low rate of infusion and increasing the rate of infusion of drug and increasing the flow rate of high flow breathing gas without allowing drug solution to accumulate in the reservoir in the BEJN.

9. The system of claim 1, wherein the patient is treated for a period of 30 minutes to 3 days.

10. The system of claim 1, wherein the patient is treated for a period of two hours to 24 hours.

11. A method for enhanced aerosol drug delivery during high flow nasal cannula therapy comprising the system of claim 1.

12-19. (canceled)
